(19) United States
(12) Patent Application Publication
(10) Pub. No.: US 2023/0202963 A1
(43) Pub. Date: Jun. 29, 2023

(54) ESTERS AS FRAGRANCE COMPOUNDS

(71) Applicant: SYMRISE AG, Holzminden (DE)

(72) Inventors: Vijayanand CHANDRASEKARAN, Holzminden (DE); Bernd HOELSCHER, Halle (DE)

(21) Appl. No.: 17/995,337

(22) PCT Filed: Apr. 8, 2020
(86) PCT No.: PCT/EP2020/060061

§ 371 Date: Oct. 3, 2022

(51) Int. Cl. C07C 69/38; C07C 69/003; C07C 69/22; C11B 9/00

(52) U.S. Cl. CPC C07C 69/38; C07C 69/003; C07C 69/22; C11B 9/0034

(57) ABSTRACT

The present invention relates to novel esters of formula (I) as defined below and their use as fragrance or malodour reducing agents. Moreover, the present invention relates to fragrance compositions comprising a compound or a mixture of compounds of formula (I) and one or more further fragrance substance(s). The invention also provides a method for perfuming a product with a compound or a mixture of compounds of formula (I) as defined below and the perfumed product as well as a method for perfuming hair, skin, textile fibers, surfaces and/or ambient air with a compound or a mixture of compounds of formula (I) as defined below.

The present invention relates to novel esters of formula (I) as defined below and their use as fragrance or malodour reducing agents. Moreover, the present invention relates to fragrance compositions comprising a compound or a mixture of compounds of formula (I) and one or more further fragrance substance(s). The invention also provides a method for perfuming a product with a compound or a mixture of compounds of formula (I) as defined below and the perfumed product as well as a method for perfuming hair, skin, textile fibers, surfaces and/or ambient air with a compound or a mixture of compounds of formula (I) as defined below.

Despite a large number of available fragrance substances, there is a constant need for novel compounds and combinations of compounds in the perfume and fragrance industry. In order to satisfy customers' needs, new fragrance profiles and complex olfactory impressions have to be developed on a regular basis. The industry is therefore constantly searching for new compounds, which exhibit special sensory properties and can serve as a basis for the composition of novel perfumes and fragrances. Such compounds should not only provide attractive olfactory impressions by themselves but also integrate harmoniously into fragrance compositions while adding further complexity to the overall impression.

A further aspect, which is highly sought-after in the fragrance and perfume industry, is the ability of fragrance compounds and compound mixtures to mask and/or reduce malodours such as body odours or unpleasant side notes caused by a fragrance compound or occurring in a fragrance composition.

The search for suitable substances that led to the present invention was complicated by the following facts:
The mechanisms of olfactory perception are not sufficiently known;
the connections between the specific olfactory perception on the one hand and the chemical structure of the associated fragrance substance on the other hand have not been sufficiently researched;
often even minor changes in the structural set-up of a known fragrance substance cause major changes in the sensory properties and affect the tolerance for the human organism.

Particularly desirable olfactory impressions for various applications in the perfume industry are musky notes, in particular in combination with woody and fruity impressions.

WO03082799A1 discloses alicyclic esters of a general formula (I) and their sensory properties, namely as musky fragrances. WO02096852A1 relates to cycloalkane carboxylic acid derivatives of a general formula (I) as fragrants with musk characteristics. Certain esters comprising a 3,3-dimethyl-1-cylopentyl or 3,3-dimethyl-1-cyclohexyl group with a musky fragrance are described in EP0472966A1. Also WO2000014051A1 deals with esters carrying cyclohexyl groups, which provide a musky odor. Several (3,3 or 5,5)dimethylcyclohex(en)yl derivatives are disclosed as malodour neutralizers in US20100209378A1. According to WO2008049257A1, 3,3-dimethylcyclohex(en)yl derivatives can be combined with certain alkenyl esters to provide a synergistic malodour counteracting effect.

The primary objective of the present invention was to find new substances that have an interesting sensory profile and are suitable as fragrance and/or malodour reducing substances for use in perfumery. In particular, musky, woody and fruity notes and combinations thereof should be provided.

In addition, fragrance substances fulfilling this primary objective should preferably have additional positive secondary properties in addition to their primary, i.e. olfactory properties, properties, such as e.g. high stability under certain application conditions, high yield, good adhesion, high substantivity or odour enhancing properties (so-called booster or enhancer effect) and/or, in combination with other fragrance and/or flavour substances, round off their naturalness, freshness, fullness, (radiant) power and/or radiance so that remarkable sensory effects can be achieved.

The above described objectives are met by a compound or mixture of compounds of formula (I)

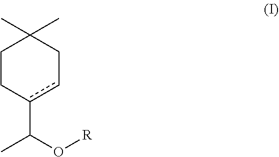

wherein R represents (CO)CH₂—COOEt, (CO)CH₂—O—CO-Et, C(CH₃)₂—CH₂—O—COOEt or COEt.

In particular, the compound(s) of formula (I) is/are selected from the group consisting of

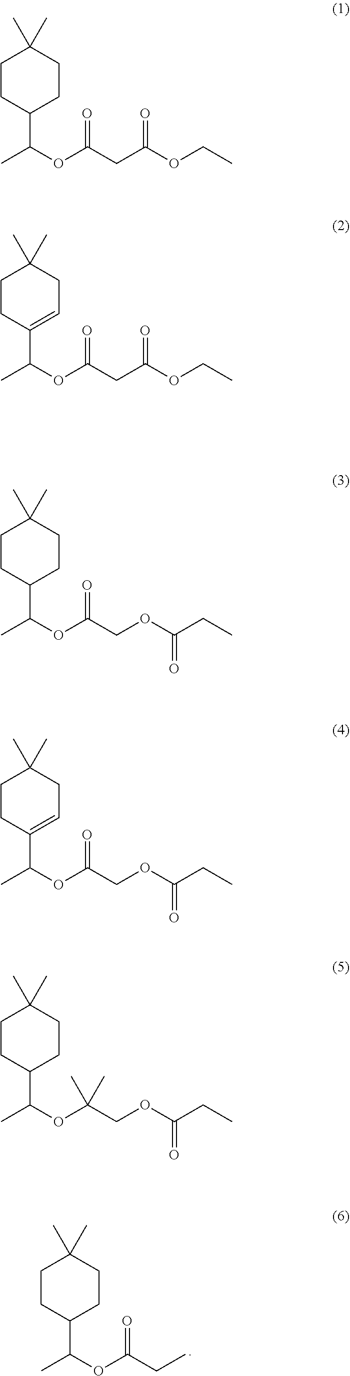 [SMILES: CCOC(=O)CC(=O)OC(C)C1CCC(C)(C)CC1 ; CCOC(=O)CC(=O)OC(C)C1=CCC(C)(C)CC1 ; CCC(=O)OCC(=O)OC(C)C1CCC(C)(C)CC1 ; CCC(=O)OCC(=O)OC(C)C1=CCC(C)(C)CC1 ; CCC(=O)OCC(C)(C)OC(C)C1CCC(C)(C)CC1 ; CCC(=O)OC(C)C1CCC(C)(C)CC1]

The compounds of formula (I) represent novel substances, which—in the context of the present invention—were found to provide interesting and attractive fragrance profiles as well as malodour counteracting effects. The olfactory properties make the compounds of the invention suitable for application in various fragrance and perfume products. For the reasons given above, the olfactory properties of the compounds according to the invention could not have been predicted. In particular, the combination of different olfactory notes was surprising. Methods to obtain the compounds are given in the examples below.

In one aspect, the present invention relates to the use of a compound or mixture of compounds as defined above as a fragrance or malodour reducing agent.

A “malodour reducing agent” in the context of the present invention is a substance or compound, which may have a pleasant fragrance itself and which is able to mask and/or reduce any unpleasant smell so that in the presence of the malodour reducing agent the unpleasant smell is no longer perceived or is perceived to a lesser degree. An unpleasant smell or malodour can be e.g. body odour or any odour present in ambient air, such as stale air, cooking smells or the smell of waste. Moreover, an unpleasant smell or malodour can also be caused as a side effect of a fragrance substance or mixture, which is used for its otherwise desirable olfactory properties.

Testing of the compounds (1) to (6) revealed the fragrance profiles as shown in table 1.

TABLE 1
Fragrance profiles of compounds (1) to (6)

| | | |
|---|---|---|
| | (1) | |
| 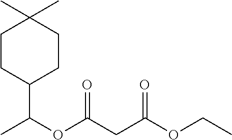 [SMILES: CCOC(=O)CC(=O)OC(C)C1CCC(C)(C)CC1] | | strawberry, anise |
| | (2) | |
| 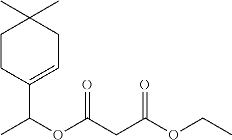 [SMILES: CCOC(=O)CC(=O)OC(C)C1=CCC(C)(C)CC1] | | woody, powdery, musky |
| | (3) | |
| 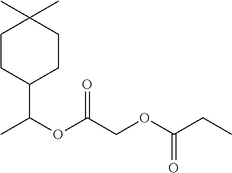 [SMILES: CCC(=O)OCC(=O)OC(C)C1CCC(C)(C)CC1] | | fruity, musky |
| | (4) | |
| 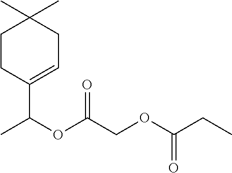 [SMILES: CCC(=O)OCC(=O)OC(C)C1=CCC(C)(C)CC1] | | pear, woody, musky |
| | (5) | |
| 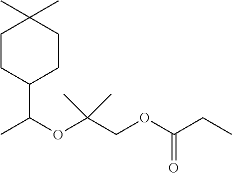 [SMILES: CCC(=O)OCC(C)(C)OC(C)C1CCC(C)(C)CC1] | | fruity, musky, woody |
| | (6) | |
| 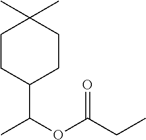 | | fruity, sweet |

In a preferred embodiment of the use described above, the compound or mixture of compounds is used for imparting, enhancing and/or modifying one or more olfactory notes selected from the group consisting of fruity, musky, woody, powdery, sweet, pear, strawberry and anise.

According to a further preferred embodiment of the use described above, compound (1) is used to impart, enhance and/or modify a strawberry and/or anise note and/or compound (2) is used to impart, enhance and/or modify a woody and/or powdery and/or musky note and/or compound (3) is used to impart, enhance and/or modify a fruity and/or musky note and/or compound (4) is used to impart, enhance and/or modify a pear and/or woody and/or musky note and/or compound (5) is used to impart, enhance and/or modify a fruity and/or musky and/or woody note and/or compound (6) is used to impart, enhance and/or modify a fruity and/or sweet note.

In another aspect, the present invention provides a fragrance composition comprising or consisting of a compound or mixture of compounds as defined above and one or more further fragrance substances.

The compounds of formula (I) according to the invention may be used together with other fragrance substances. Such fragrance substance compositions may be prepared in the usual way, for example by simple mixing or homogenisation of the ingredients. These further fragrance substances can be any other fragrance substances. Examples of fragrance that can be advantageously combined with the compound of formula (I) within the scope of the present invention can be found, for example, in S. Arctander, Perfume and Flavor Materials, Vol. I and II, Montclair, N.J. 1969, Eigenverlag, or K. Bauer et al., Common Fragrance and Flavor Materials, 4th Edition, Wiley-VCH, Weinheim 2001.

The fragrance compositions according to the invention may be adsorbed to a carrier which ensures both a fine distribution of the fragrance substances in a product and a controlled release during application. Such carriers may be porous inorganic materials such as light sulphate, silica gels, zeolites, gypsums, clays, clay granules, gas concrete, etc. or organic materials such as wood, cellulose-based materials, sugars, dextrins (e.g. maltodextrin) or plastics such as PVC, polyvinyl acetates or polyurethanes. The composition according to the invention and a carrier may represent a perfumed product according to the invention (as described below).

Fragrance compositions or products according to the invention may also be present in microencapsulated form, spray-dried form, as inclusion complexes or as extrusion products and—in case of a fragrance composition—may be added in this form to a product to be perfumed (as described herein below).

If applicable, the properties of such modified compositions or products can be further optimised by so-called “coating” with suitable materials in view of a more targeted release of fragrance, preferably using wax-like plastics such as e.g. polyvinyl alcohol. The resulting products in turn are products according to the invention.

Microencapsulation can, for example, be achieved by the so-called coacervation process with the aid of capsule materials, e.g. polyurethane-like substances or soft gelatine. Spray-dried products are preferably produced by spray-drying an emulsion or dispersion containing the fragrance composition, whereby modified starches, proteins, dextrins and vegetable gums can be used as carriers. Inclusion complexes can be prepared e.g. by incorporating dispersions of the fragrance composition and cyclodextrins or urea derivatives into a suitable solvent, e.g. water. Extrusion products can be obtained e.g. by fusing the fragrance compositions with a suitable wax-like substance and by extrusion followed by solidification, if applicable in a suitable solvent, e.g. isopropanol.

According to a further aspect, the present invention relates to a method for perfuming a product and/or for reducing malodour in a product comprising the following steps:
(i) providing a compound or mixture of compounds as described above or a fragrance composition as described above, and
(ii) adding the compound or mixture of compounds or the fragrance composition to the product to be perfumed, preferably in a sensorially effective amount, in particular in an amount sufficient to impart, enhance and/or modify one or more olfactory notes selected from the group consisting of fruity, musky, woody, powdery, sweet, pear, strawberry and anise and/or in an amount sufficient to reduce malodour.

A sensorially effective amount refers to a total amount of the compound or mixture of compounds according to the invention, which exerts a sensorial effect. The sensorial effect can be imparting, enhancing and/or modifying certain olfactory notes and/or masking and/or reducing unpleasant notes (malodour). Thus, a sensorially effective amount is an amount, in which the sensorial effect of the compound or mixture of compounds can be perceived in the product by a user in comparison to a product, which does not comprise the compound or mixture of compounds of the present invention. Suitable amounts to provide this effect are given below in the context of the perfumed product.

In a preferred embodiment of the method described above, in step (ii) compound (1) is added to the product to be perfumed to impart, enhance and/or modify a strawberry and/or anise note and/or compound (2) is added to the product to be perfumed to impart, enhance and/or modify a woody and/or powdery and/or musky note and/or compound (3) is added to the product to be perfumed to impart, enhance and/or modify a fruity and/or musky note and/or compound (4) is added to the product to be perfumed to impart, enhance and/or modify a pear and/or woody and/or musky note and/or compound (5) is added to the product to be perfumed to impart, enhance and/or modify a fruity and/or musky and/or woody note and/or compound (6) is added to the product to be perfumed to impart, enhance and/or modify a fruity and/or sweet note.

In another aspect, the present invention also relates to a perfumed product comprising a compound or a mixture of compounds as defined above or a fragrance composition as defined above. Preferably, the product is obtained by a method for perfuming a product as described above.

In one embodiment, the perfumed product described above comprises one or more additive(s), excipient(s) and/or active substances.

The additives, excipients and/or active substances are preferably not fragrance substances and, are preferably selected from the group consisting of: preservatives, preferably those mentioned in US 2006/0089413, abrasives, anti-acne agents and sebum reducing agents, preferably those mentioned in WO 2008/046791, anti-aging agents, preferably those mentioned in WO 2005/123101, antibacterial agents, anti-cellulite agents, anti-dandruff agents, preferably those mentioned in WO 2008/046795, anti-inflammatory agents, irritation-preventing agents, anti-irritants (anti-inflammatory, irritation-inhibiting and irritation-preventing agents), preferably those mentioned in WO 2007/042472 and US 2006/0089413, antimicrobial agents, preferably those mentioned in WO 2005/123101, antioxidants, preferably those mentioned in WO 2005/123101, astringents, antiseptic agents, antistatic agents, binders, buffers, carrier materials, preferably those mentioned in WO 2005/123101, chelating agents, preferably those mentioned in WO 2005/123101, cell stimulants, cleansing agents, caring agents, depilatories, surfactants, deodorising agents and antiperspirants, preferably those mentioned in WO 2005/123101, plasticizers, emulsifiers, preferably those mentioned in WO 2005/123101, enzymes, essential oils, preferably those mentioned in US 2008/0070825, insect repellents, preferably those mentioned in WO 2005/123101, fibres, film-forming agents, (further) fixers, foaming agents, foam stabilisers, substances for the prevention of foaming, foam boosters, fungicides, gelling agents and gel-forming agents, preferably those mentioned in WO 2005/123101, hair care products, hair deforming agents, hair straightening agents, moisture regulators (moisturising and/or wetting agents and/or humectants), preferably those mentioned in WO 2005/123101, osmolytes, preferably those mentioned in WO 2005/123101, compatible solutes, preferably those mentioned in WO 01/76572 and WO 02/15686, bleaching agents, strengthening agents, stain removing agents, optical brightening agents, impregnating agents, soil repellents, friction reducing agents, lubricants, moisturizing creams, ointments, opacifiers, plasticising agents, covering agents, polish, brighteners, polymers, preferably those mentioned in WO 2008/046676, powders, proteins and protein hydrolysates, preferably those mentioned in WO 2005/123101 and WO 2008/046676, refattening agents, abrasive agents, skin-soothing agents, skin-cleansing agents, skin-care agents, skin repair agents, preferably containing cholesterol and/or fatty acids and/or ceramides and/or pseudoceramides, thereby preferably those mentioned in WO 2006/053912, skin whitening agents, preferably those mentioned in WO 2007/110415, skin protecting agents, skin softeners, skin cooling agents, preferably those mentioned in WO 2005/123101, skin warming agents, preferably those mentioned in WO 2005/123101, stabilisers, UV-absorbing agents and UV-filters, preferably those mentioned in WO 2005/123101, benzylidene-beta-dicarbonyl compounds, preferably those mentioned in WO 2005/107692, alpha-benzoyl cinnamic acid nitriles, preferably those mentioned in WO 2006/015954, AhR receptor antagonists, preferably those mentioned in WO 2007/128723 and WO 2007/060256, detergents, fabric softeners, suspending agents, skin tanning agents, preferably those mentioned in WO 2006/045760, thickeners, vitamins, preferably those mentioned in WO 2005/123101, fatty oils, waxes and fats, preferably those mentioned in WO 2005/123101, phospholipids, preferably those mentioned in WO 2005/123101, fatty acids (saturated fatty acids, mono- or polyunsaturated fatty acids, α-hydroxy acids, polyhydroxy fatty acids), preferably those mentioned in WO 2005/123101, dyes and colour protecting agents as well as pigments, preferably those mentioned in WO 2005/123101, anticorrosives, alcohols and polyols, preferably those mentioned in WO 2005/123101, surfactants, preferably those mentioned in WO 2005/123101, animal extracts, yeast extracts, extracts of algae or microalgae, electrolytes, liquefiers, organic solvents, preferably those mentioned in WO 2005/123101, hair growth modulating agents (hair growth promoting or hair growth inhibiting), preferably those mentioned in EP 2168570 and EP 2193785 or silicones and silicone derivatives, preferably those mentioned in WO 2008/046676, preferably selected from the group consisting of preservatives, inorganic salts, chelating agents, surfactants, skin and/or hair caring agents, enzymes, emulsifiers, fats, fatty oils, waxes, fatty alcohols, silicones, silicone derivatives and water.

In a preferred embodiment, the product is selected from the group consisting of detergents and cleaning agents, hygiene or care products, preferably in the field of body and hair care, cosmetics and household, preferably from the group consisting of perfume extracts, eau de parfums, eau de toilettes, aftershave lotions, eau de colognes, pre-shave products, splash colognes, perfumed refreshing wipes, acidic, alkaline or neutral detergents, textile fresheners, ironing aids, liquid detergents, powder detergents, laundry pretreatments, fabric softeners, washing soaps, washing tablets, disinfectants, surface disinfectants, air fresheners, aerosol sprays, waxes and polishes, body care products, hand creams and lotions, foot creams and lotions, depilatory creams and lotions, after-shave creams and lotions, tanning creams and lotions, hair care products, deodorants, antiperspirants, decorative cosmetic products, candles, lamp oils, incense sticks, insecticides, repellents and fuels.

In a further preferred embodiment, in the product described above, the total amount of compound(s) of formula (I), is in a range of from 0.01 to 10 wt. %, preferably 0.1 to 5 wt.-%, particularly preferably 0.25 to 3 wt.-%, in each case with respect to the total weight of the product.

In the amounts specified above, the compound or mixture of compounds of the present invention are sensorially effective and impart their characteristic fragrance profile to the product and the fragrance is perceived by a user of the product and/or the perception of malodour is reduced.

In yet another aspect, the present invention relates to a method for perfuming hair, skin, textile fibers, surfaces and/or ambient air and/or for reducing malodor of hair, skin, textile fibers, surfaces and/or ambient air comprising or consisting of the following steps
(i) providing a compound or mixture of compounds as described above, a fragrance composition as described above or a perfumed product as described above, and
(ii) applying or introducing the compound or mixture of compounds, the fragrance composition or the product to the hair or skin or fibres or surface to be perfumed, or into the ambient air to be perfumed in a sensorially effective amount, in particular in an amount sufficient to impart, enhance and/or modify one or more olfactory notes selected from the group consisting of fruity, musky, woody, powdery, sweet, pear, strawberry and anise and/or in an amount sufficient to reduce malodour.

In a preferred embodiment of the method for perfuming hair, skin, textile fibers, surfaces and/or ambient air described above, in step (ii) compound (1) is applied or introduced to impart, enhance and/or modify a strawberry and/or anise note and/or compound (2) is applied or introduced to impart, enhance and/or modify a woody and/or powdery and/or musky note and/or compound (3) is applied or introduced to impart, enhance and/or modify a fruity and/or musky note and/or compound (4) is applied or introduced to impart, enhance and/or modify a pear and/or woody and/or musky note and/or compound (5) is applied or introduced to impart, enhance and/or modify a fruity and/or musky and/or woody note and/or wherein (6) is applied or introduced to impart, enhance and/or modify a fruity and/or sweet note.

The present invention is explained in more detail using the following nonlimiting examples.

### EXAMPLE 1: PREPARATION OF PROPANEDIOATE DERIVATIVES

### General Experimental Procedure:

A mixture of alcohol (1.00 eq), Diethylmalonate (2.1 eq) and Sodium ethoxide (0.01 eq) was heated to 160° C. Ethanol was distilled/collected by reactive distillation. After completion of the reaction, at room temperature, water (2 vol) was added to reaction mixture, compound was extracted with MTBE (10 vol×2), combined organic layer washed with sat. NaCl (10 vol×2), dried with MgSO₄ and solvents were removed using rotary evaporator to get the crude compound. Crude compound was purified by Kugelrohr distillation T=180° C.; p=0.80 mbar) followed by column chromatography (2.5-5% EtOAc in Cyclohexane).

### O3-[1-(4,4-dimethylcyclohexyl)ethyl] O1-ethyl Propanedioate was Obtained in 75% Yield, >95% Purity, Starting from 1-(4,4-dimethylcyclohexyl)ethanol

¹H NMR (400 MHz, CDCl₃) δ 4.82 (p, J=6.4 Hz, 1H), 4.20 (qd, J=7.1, 2.2 Hz, 2H), 3.35 (s, 2H), 1.63-1.54 (m, 1H), 1.52-1.45 (m, 1H), 1.39 (dtt, J=10.2, 7.0, 2.9 Hz, 3H), 1.28 (t, J=7.2 Hz, 3H), 1.21 (d, J=6.4 Hz, 3H), 1.20-1.11 (m, 4H), 0.89 (s, 3H), 0.86 (s, 3H).

¹³C NMR (101 MHz, CDCl₃) δ 166.74, 166.29, 76.05, 61.45, 42.44, 42.07, 38.75, 38.69, 32.81, 29.96, 24.27, 24.13, 24.11, 17.18, 14.07.

MS: 255, 226, 208, 193, 155, 138, 115, 109, 95, 82

Odor: strawberry, anise

### O3-[1-(4,4-dimethylcyclohexen-1-yl)ethyl] O1-ethyl Propanedioate was Obtained in 65% Yield, >96% Purity, Starting from 1-(4,4-dimethylcyclohexen-1-yl)ethanol

¹H NMR (400 MHz, CDCl₃) δ 5.68-5.61 (m, 1H), 5.32 (q, J=6.6 Hz, 1H), 4.24-4.15 (m, 2H), 3.37-3.32 (m, 2H), 2.12-1.91 (m, 2H), 1.84-1.78 (m, 2H), 1.37 (t, J=6.4 Hz, 2H), 1.33 (d, J=6.6 Hz, 3H), 1.28 (t, J=7.1 Hz, 3H), 0.90 (s, 3H), 0.88 (s, 3H).

¹³C NMR (101 MHz, CDCl₃) δ 166.69, 165.95, 135.19, 123.38, 75.40, 61.44, 42.04, 38.83, 35.17, 28.58, 28.43, 27.76, 21.72, 18.76, 14.08.

MS: 268, 250, 235, 194, 177, 154, 136, 121, 93, 79, 55.

Odor: woody, powdery, musky

### EXAMPLE 2: PREPARATION OF PROPANOATE DERIVATIVES

### General Experimental Procedure:

(a) Chloroacetate derivative: To a mixture of alcohol (1.00 eq), toluene (3 vol) and pyridine (1.2 eq), below 30° C. chloroacetyl chloride (1.1 eq) was added, reaction maintained at 30° C. until completion of the reaction (2-3 h). reaction mixture was quenched at <15° C., with cold water (5 vol), organic phase was separated, washed with water (5 vol×2), sat. NaCl solution (10 vol×2), dried with Na₂SO₄ and solvents were removed using rotary evaporator to get the crude compound. Purification by bulb to bulb (Kugelrohr) distillation (˜T=141° C., p=0.81 mbar).
(b) ethoxy-2-oxo-ethyl-propanoate derivative: To a solution of chloroacetate derivative (1.0 eq) in THF (15 vol), below 40° C. slowly added propionic acid (1.5 eq) followed by portionwise addition of K2003 (2.5 eq). Reaction maintained at reflux temperature until completion of the reaction. Reaction mixture quenched with water (20 mL) and compound extracted with MTBE (10 vol×2), organic layer washed with water (5 vol×2), dried with MgSO₄, filtered and the solvents were removed using rotary evaporator to get the crude compound. Crude was purified by bulb to bulb (Kugelrohr) distillation (˜T=190° C.; p=0.85 mbar) followed by column chromatography (1:20→1:10, EtOAc in Cyclohexane).

### 1-(4,4-dimethylcyclohexyl)ethyl 2-chloroacetate was Obtained in 85% Yield, >95% Purity, Starting from 1-(4,4-dimethylcyclohexyl)ethanol

¹H NMR (400 MHz, CDCl₃) δ 4.85 (dq, J=6.4 Hz, 1H), 4.06 (d, J=14.6 Hz, 1H), 4.02 (d, J=14.7 Hz, 1H), 1.62-1.54 (m, 1H), 1.53-1.45 (m, 1H), 1.45-1.34 (m, 3H), 1.23 (d, J=6.4 Hz, 3H), 1.25-1.10 (m, 4H), 0.90 (s, 3H), 0.86 (s, 3H).

¹³C NMR (101 MHz, CDCl₃) δ 167.00, 77.03, 42.46, 41.23, 38.71, 38.65, 32.80, 29.96, 24.28, 24.10, 24.08, 17.19.

MS: 183, 165, 155, 138, 123, 95, 81, 69.

### [2-[1-(4,4-dimethylcyclohexyl)ethoxy]-2-oxo-ethyl] Propanoate was Obtained in 70% Yield, >98% Purity, Starting from 1-(4,4-dimethylcyclohexyl)ethyl 2-chloroacetate

¹H NMR (400 MHz, CDCl₃) δ 4.85 (dq, J=6.4 Hz, 1H), 4.59 (s, 2H), 2.45 (q, J=7.6 Hz, 2H), 1.58 (m, 1H), 1.47 (dtd, J=10.9, 3.6, 1.8 Hz, 1H), 1.43-1.31 (m, 3H), 1.21 (d, J=6.4 Hz, 3H), 1.19 (t, J=7.5 Hz, 3H), 1.19-1.11 (m, 4H), 0.89 (s, 3H), 0.86 (s, 3H).

¹³C NMR (101 MHz, CDCl₃) δ 173.80, 167.67, 76.05, 60.80, 42.51, 38.73, 38.69, 32.81, 29.96, 27.19, 24.26, 24.19, 24.11, 17.21, 8.98.

MS: 255, 211, 183, 154, 138, 123, 109, 95, 83

Odor: fruity, musky

### 1-(4,4-dimethylcyclohexen-1-yl)ethyl 2-chloroacetate was Obtained in 90% Yield, Starting from 1-(4,4-dimethylcyclohexen-1-yl)ethanol

¹H NMR (600 MHz, CDCl₃) δ 5.67 (tdt, J=3.7, 2.4, 1.5 Hz, 1H), 5.38-5.32 (m, 1H), 4.04 (s, 2H), 2.08-1.95 (m, 2H), 1.82 (tdt, J=18.2, 14.8, 3.1 Hz, 2H), 1.38 (ddd, J=7.4, 6.2, 1.6 Hz, 2H), 1.35 (d, J=6.6 Hz, 3H), 0.90 (s, 3H), 0.88 (s, 3H).

¹³C NMR (151 MHz, CDCl₃) δ 166.63, 134.96, 123.93, 76.40, 41.25, 38.81, 35.12, 28.58, 28.52, 27.65, 21.65, 18.78.

MS: 230, 215, 193, 181, 161, 136, 121, 107, 93, 79, 67, 55, 41, 29

### [2-[1-(4,4-dimethylcyclohexen-1-yl)ethoxy]-2-oxo-ethyl] Propanoate was Obtained in 60% Yield, >98% Purity, Starting from 1-(4,4-dimethylcyclohexen-1-yl)ethyl 2-chloroacetate

¹H NMR (400 MHz, CDCl₃) δ 5.64 (qd, J=3.8, 3.0, 1.3 Hz, 1H), 5.34 (q, J=6.5 Hz, 1H), 4.62 (d, J=15.9 Hz, 1H), 4.57 (d, J=15.9 Hz, 1H), 2.44 (q, J=7.6 Hz, 2H), 2.09-1.90 (m, 2H), 1.88-1.73 (m, 2H), 1.37 (t, J=6.4 Hz, 2H), 1.33 (d, J=6.5 Hz, 3H), 1.18 (t, J=7.6 Hz, 3H), 0.89 (s, 3H), 0.88 (s, 3H).

¹³C NMR (101 MHz, CDCl₃) δ 173.77, 167.33, 135.08, 123.67, 75.49, 60.79, 38.81, 35.14, 28.58, 28.48, 27.70, 27.18, 21.67, 18.82, 8.98.

MS: 268, 250, 194, 154, 136, 121, 93

Odor: pear, woody, musky

### 2-[1-(4,4-dimethylcyclohexyl)ethoxy]-2-methyl-propyl] Propanoate

2-[1-(4,4-dimethylcyclohexyl)ethoxy]-2-methyl-propyl] propanoate was prepared according to EP1262474.

### a) 2-[1-(4,4-dimethylcyclohexyl)ethoxy]-2-methyl-propan-1-ol: According to EP1262474

¹H NMR (600 MHz, CDCl₃) δ 3.49-3.43 (m, 1H), 3.41-3.35 (m, 2H), 2.19 (t, J=6.3 Hz, 1H, —OH), 1.61-1.57 (m, 1H), 1.53-1.48 (m, 1H), 1.44-1.41 (m, 1H), 1.41-1.40 (m, 1H), 1.24-1.12 (m, 11H), 1.10 (d, J=6.2 Hz, 3H), 0.90 (s, 3H), 0.87 (s, 3H).

¹³C NMR (151 MHz, CDCl₃) δ 75.13, 71.19, 71.03, 44.97, 39.30, 39.23, 33.03, 30.14, 25.08, 24.19, 24.16, 23.07, 22.76, 19.87.

MS: 228, 197, 139, 117, 97, 83, 73, 59.

### b) [2-[1-(4,4-dimethylcyclohexyl)ethoxy]-2-methyl-propyl] Propanoate

¹H NMR (400 MHz, CDCl₃) δ 3.95 (s, 2H), 3.44 (p, J=5.8 Hz, 1H), 2.36 (q, J=7.6 Hz, 2H), 1.63-1.59 (m, 1H), 1.52-1.47 (m, 1H), 1.41-1.35 (m, 2H), 1.19 (s, 3H), 1.19 (s, 3H), 1.18 (s, 2H), 1.16 (t, J=7.6 Hz, 3H), 1.16-1.08 (m, 3H), 1.08 (d, J=6.2 Hz, 3H), 0.88 (s, 3H), 0.85 (s, 3H).

¹³C NMR (101 MHz, CDCl₃) δ 174.35, 73.78, 71.55, 70.40, 44.94, 39.32, 39.29, 33.04, 30.11, 27.69, 25.11, 24.28, 24.16, 24.12, 23.80, 20.01, 9.17.

MS: 269, 245, 197, 129, 97, 83, 69, 57

Odor: fruity, musky, woody

### 1-(4,4-dimethylcyclohexyl)ethyl Propanoate

¹H NMR (400 MHz, CDCl₃) δ 4.80 (p, J=6.4 Hz, 1H), 2.34 (q, J=7.4 Hz, 2H), 1.65-1.58 (m, 1H), 1.56-1.49 (m, 1H), 1.42 (ddd, J=10.2, 4.0, 1.9 Hz, 2H), 1.42-1.33 (m, 1H), 1.30-1.14 (m, 4H), 1.20 (d, J=6.4 Hz, 3H), 1.17 (t, J=7.6 Hz, 3H), 0.92 (s, 3H), 0.89 (s, 3H).

¹³C NMR (101 MHz, CDCl₃) δ 174.18, 74.25, 42.60, 38.82, 38.77, 32.85, 29.99, 28.00, 24.39, 24.20, 24.14, 17.37, 9.31.

MS: 168, 138, 123, 109, 69, 57, 41, 29

Odor: fruity, sweet

### EXAMPLE 3; SENSORY ANALYSIS OF THE MALODOR COUNTERACTING EFFECT

The compounds of formula (I) were found to provide a malodour counteracting effect. Of all the compounds tested, compound (1) showed the best malodour counteracting effect, i.e. highly significant more perfume and lower malodour intensity.

Kitchen Malodor Vs formula 1 (3.7:4.0); Sweat Malodor Vs Formula 1 (2.6:3.6); smoke malodour Vs Formula 1 (2.9:4.6). The numbers in parentheses represent the compared intensity.

Magnitude Scale: (0-10; no odor-strongest); 1-5 μl sample and 5-10 μl standard malodor solutions were used for analysis.

1. A compound or mixture of compounds of formula (I) 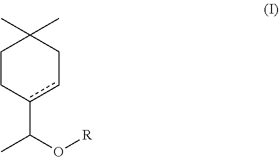
wherein R represents (CO)CH₂—COOEt, (CO)CH₂—O—CO-Et, C(CH₃)₂—CH₂—O—COOEt or COEt.

2. The compound or mixture of compounds of claim 1, wherein the compound or the mixture of compounds are selected from:

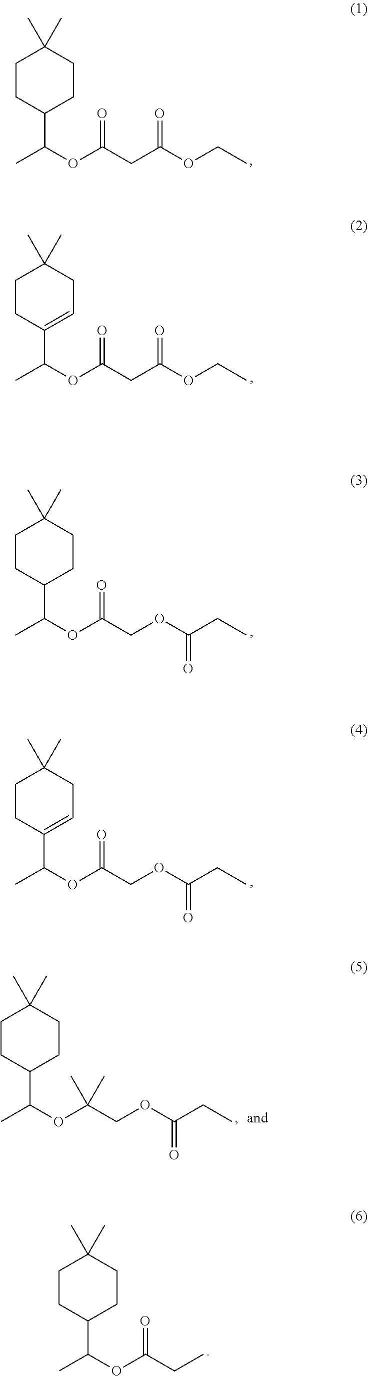 [SMILES: CCOC(=O)CC(=O)OC(C)C1CCC(C)(C)CC1 ; CCOC(=O)CC(=O)OC(C)C1=CCC(C)(C)CC1 ; CCC(=O)OCC(=O)OC(C)C1CCC(C)(C)CC1 ; CCC(=O)OCC(=O)OC(C)C1=CCC(C)(C)CC1 ; CCC(=O)OCC(C)(C)OC(C)C1CCC(C)(C)CC1 ; CCC(=O)OC(C)C1CCC(C)(C)CC1]

3-6. (canceled)

7. A method for perfuming or reducing malodor of a product comprising:
providing the compound or the mixture of compounds of claim 1; and
(ii) adding the compound or the mixture of the compounds to the product.

8. The method of claim 7, wherein:
compound (1) is added to the product and imparts or enhances a strawberry and/or anise note,
compound (2) is added to the product and imparts or enhances a woody and/or powdery and/or musky note,
compound (3) is added to the product and imparts or enhances a fruity and/or musky note,
compound (4) is added to the product and imparts or enhances a pear and/or woody, and/or musky note,
compound (5) is added to the product and imparts or enhances a fruity and/or musky and/or woody note, and/or
compound (6) is added to the product and imparts or enhances a fruity and/or sweet note.

9. A perfumed product comprising the compound or the mixture of the compounds of claim 1.

10. The perfumed product of claim 9, selected from detergents and cleaning agents, hygiene or care products textile fresheners, ironing aids, liquid detergents, powder detergents, laundry pre-treatments, fabric softeners, washing soaps, washing tablets, disinfectants, surface disinfectants, air fresheners, aerosol sprays, waxes and polishes, body care products, hand creams and lotions, foot creams and lotions, depilatory creams and lotions, after-shave creams and lotions, tanning creams and lotions, hair care products, deodorants, antiperspirants, decorative cosmetic products, candles, lamp oils, incense sticks, insecticides, repellents and fuels.

11. The perfumed product of claim 9, wherein the compound or the mixture of compounds is in a total amount of from 0.01 to 10 wt. %, with respect to the total weight of the product.

12. A method for perfuming hair, skin, textiles, surfaces, or ambient air and/or reducing malodor of the hair, skin textiles, surfaces, or ambient air comprising
(i) providing the compound or the mixture of compounds of claim 1; and
(ii) applying or introducing a sensorially effective amount of the compound or the mixture of compounds to the hair, skin, textiles, surfaces, or ambient air and imparting or enhancing one or more olfactory notes selected from fruity, musky, woody, powdery, sweet, pear, strawberry, and anise to the hair, skin, textiles, surfaces, or ambient air.

13. The method of claim 12, wherein:
compound (1) is applied or introduced and imparts or enhances a strawberry and/or anise note,
compound (2) is applied or introduced and imparts or enhances a woody and/or powdery and/or musky note,
compound (3) is applied or introduced and imparts or enhances a fruity and/or musky note,
compound (4) is applied or introduced and imparts or enhances a pear and/or woody and/or musky note,
compound (5) is applied or introduced and imparts or enhances a fruity and/or musky and/or woody note, and/or
compound (6) is applied or introduced and imparts or enhances a fruity and/or sweet note.

14. The compound of the mixture of compounds of claim 1 comprising the compound of formula (1):

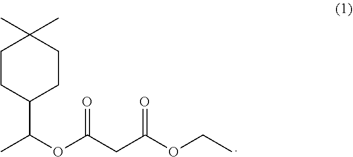 [SMILES: CCOC(=O)CC(=O)OC(C)C1CCC(C)(C)CC1]

15. The compound or the mixture of compounds of claim 1 comprising the compound of formula (2):

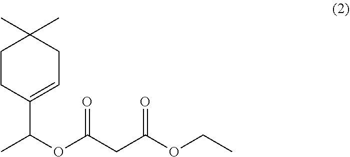 [SMILES: CCOC(=O)CC(=O)OC(C)C1=CCC(C)(C)CC1]

16. The compound or the mixture of compounds of claim 1 comprising the compound of formula (3):

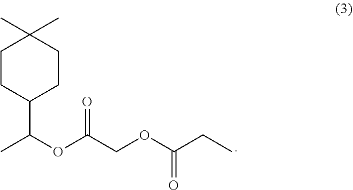 [SMILES: CCC(=O)OCC(=O)OC(C)C1CCC(C)(C)CC1]

17. The compound or the mixture of compounds of claim 1 comprising the compound of formula (4):

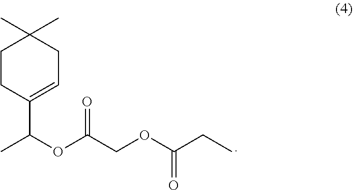 [SMILES: CCC(=O)OCC(=O)OC(C)C1=CCC(C)(C)CC1]

18. The compound or the mixture of compounds of claim 1 comprising the compound of formula (5):

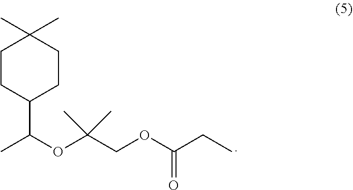 [SMILES: CCC(=O)OCC(C)(C)OC(C)C1CCC(C)(C)CC1]

19. The compound or the mixture of compounds of claim 1 comprising the compound of formula (6):

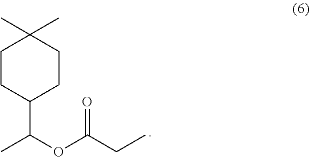

20. The perfumed product of claim 9, wherein the compound or the mixture of compounds is in a total amount of from 0.1 to 5 wt.-%, with respect to the total weight of the product.

21. The perfumed product of claim 9, wherein the compound or the mixture of compounds is in a total amount of 0.25 to 3 wt.-%, with respect to the total weight of the product.
